(19) United States
(12) Patent Application Publication
(10) Pub. No.: US 2006/0161454 A1
(43) Pub. Date: Jul. 20, 2006

(54) Doctor express care card

(76) Inventor: Althea Vernice Hankins, Gwynedd Valley, PA (US)

Correspondence Address: Althea Hankins MD FACP, PO Box 9959, Philadelphia, PA, 19118-0959, US

(21) Appl. No.: 11/183,437

(22) Filed: Jul. 18, 2005

Related U.S. Application Data
(60) Provisional application No. 60/588,965, filed on Jul. 19, 2004.

(51) Int. Cl. G06Q 10/00; A61B 5/00

(52) U.S. Cl. 705/2; 600/300

(57) ABSTRACT

The Doctor's Express Care Card is a health processing method that includes an all inclusive payment and alternative health care delivery system. The providers agree to accept negotiated fees for recognized CPT Codes, (Physicians' Current Procedural Terminology-CPT 2005). This new and unique process is a useful solution to the health care crisis. I encompass ambulatory utilization for illness prevention and control while guaranteeing fair fee for service payment to providers that currently may be forced into receiving only $6.00 a month for out-patient care. This is one of the reasons doctors are leaving the profession. The Doctors Express Care Card will reverse that trend-hence force the name. Ambulatory care accessibility will prevent the over utilization of emergency rooms and poor clinical out comes for the uninsured. Most can afford the Card's “assurance” coverage. The State of Pennsylvania paid $580 million in uninsured care in 2004 alone.

### CROSS-REFERENCE TO RELATED APPLICATIONS

A provisional utility patent was granted No. 60/588,965.

### STATEMENT REGARDING FEDERALLY SPONSERED RESEARCH OR DEVELOPMENT

Does not apply. Private.

### BACKGROUND OF THE INVENTION

The Doctor Express Care Card solves the problem of the uninsured population of America by creating a new health assurance delivery system.

As a person in the private practice of Internal Medicine, and a former director of a Medicare “HMO” system, “Union Med”, which I created and received certification for, and former Medicare consultant for AmeriChoice Insurance Company, I was in the unique situation to create a new health care delivery system.

I saw the problem from both sides.

The Doctor's Express Care Card creates an expansion of medical care while providing fair reimbursement for the Providers.

Currently, insurance companies, especially HMO's are top heavy with administration and low with reimbursement. Physicians and providers are leaving the practice of medical care, especially primary care. The cost and qualifications of standard delivery systems is keeping 44 million individuals from routine medical access. This results in substandard clinical outcomes, emergency room over-utilization, and more expense in health care dollars for the country at large.

The State of Pennsylvania is so over whelmed they have considered cutting access, by limiting appointments and needed medications. The State of New Jersey will consider using our system once a full patent is granted.

### SUMMARY OF THE INVENTION

The Card provides utilization units that are based on $25.00 increments. Patients can decide how to mix and match services. Providers would agree to a standardized fee agreement. The fees would be based on a fair industry standard, and at least three time HMO rates and at least 50% of the Medicare fee schedule. The cards would start at $250.00 and $25.00 only for administration. All provider fees are placed in a secure account for utilization by the card holder only. Unlike insurance companies, we will NOT invest the patients money, leaving them and the providers without money or services when the company closes.

This unique concept will open up a variety of care to a broad section of people.

### DETAILED DESCRIPTION OF THE INVENTION

Each card will be manufactured with a unique number and activation code. The units per service will be predetermined per CPT standard. The provider will be verified and the units of service determined per CPT Code. The correct address for fee delivery will be ascertained.

There are no provider panels with this new model. This is an expansion of fee-for-service care.

Patients will have access to Providers that accept the card through the web site or 800 telephone information numbers.

The services are freely transferable though the card.

Primary care physicians will receive a “Welcome from Doctor Express” urine dip stick jar to screen twenty patients for at least ten different disease states.

Claims

This new health care delivery system increases and makes affordable, health care with a variety of Providers. It also providers free routine screening and addresses the economic concerns of care givers.

1. The “assurance” is on the card.

2. The health care delivery system travels with the card.

3. There are several denomination available based on $25.00 delivery units minus the $25.00 administrative fee.

4. The fee payment schedule is based on median/average Nation reimbursement as possible for CPT code.

5. This Card is for ambulatory non critical care only.

6. The Card encourages illness treatment and prevention by a free urinary screening program to the initial primary care provider. With less than three minutes, the provider will be able to screen for up to twelve diseases for ten patients. This will assist with the early detection of many chronic medical problems.

7. The pharmacy denomination will include brand name medications for full price based on a generic fee schedule.
